SEQUENCE LISTING (8 sequences)

SEQ ID NO: 1 - DNA, 16 bp, Artificial Sequence
  Synthetic Sequence
gacgatgagtcctgag

SEQ ID NO: 2 - DNA, 14 bp, Artificial Sequence
  Synthetic Sequence
tactcaggactcat

SEQ ID NO: 3 - DNA, 17 bp, Artificial Sequence
  Synthetic Sequence
ctcgtagactgcgtacc

SEQ ID NO: 4 - DNA, 18 bp, Artificial Sequence
  Synthetic Sequence
agctggtacgcagtctac

SEQ ID NO: 5 - DNA, 17 bp, Artificial Sequence
  Synthetic Sequence
gactgcgtaccagctta

SEQ ID NO: 6 - DNA, 17 bp, Artificial Sequence
  Synthetic Sequence
gatgagtcctgagtaaa

SEQ ID NO: 7 - DNA, 17 bp, Artificial Sequence
  Synthetic Sequence
ctgcgtaccagcttagc

SEQ ID NO: 8 - DNA, 17 bp, Artificial Sequence
  Synthetic Sequence
tgagtcctgagtaaagc

(19) United States
(12) Plant Patent Application Publication
(10) Pub. No.: US 2016/0183432 P1
(43) Pub. Date: Jun. 23, 2016

(54) Nepeta cataria L. var. "Thaya"

(50) ;

(71) Applicant: E I DU PONT DE NEMOURS AND COMPANY, Wilmington, DE (US)

(72) Inventors: Thaya Silke Ganzke, Garnet Valley, PA (US); David L. Hallahan, Wilmington, DE (US)

(21) Appl. No.: 14/544,332

(22) Filed: Dec. 22, 2014

(51) Int. Cl. A01H 5/00

(52) U.S. Cl. PLT/258

(57) ABSTRACT

A new and distinct hardy perennial Catmint plant, Nepeta “Thaya” characterized at least by high oil yield.

### Field of the Invention

The subject matter disclosed herein relates to a new and distinct form of catmint (Nepeta cataria L.), useful in the production of essential oil.

### Background

The catmint (catnip) plant Nepeta cataria L. has historically been used in tea and herbal remedies, and has long been known for its excitatory effect on cats (Family Felidae). The species has thus been grown primarily for leaf to satisfy these markets.

The oil extracted from Nepeta cataria L. contains a high proportion of iridoid monoterpenes, particularly nepetalactones, which are the active constituents of the oil that contribute to its excitatory effects in cats. The oil is also known for its insect repellent properties. Additionally, catmint oil can be used as a raw material for the production of dihydronepetalactones, which are also effective insect repellents. The utility of catmint for most of the aforementioned uses is dependent on the quantity of oil produced. Thus it would be beneficial to have a catmint plant variety with high oil content. To date, no isolation or breeding for high oil yielding varieties of catmint appears to have been reported.

### SUMMARY OF INVENTION

The present invention relates to a new and distinct cultivar of Nepeta cataria, designated, “Thaya”. Nepeta is in the family Lamiaceae. This plant was selected by the inventors from commercially available Nepeta cataria seed. The cultivar “Thaya” is characterized by high oil yields (up to 0.33% on a dry weight basis) as compared to yields of 0.1-0.2% obtained from a control population. The new variety has been reproduced by asexual propagation (by cuttings). Each of the progeny exhibits identical characteristics to the original plant. Asexual propagation shows that the characteristics and distinctions of the cultivar come true to form and are established and transmitted through succeeding propagations.

The present invention has not been evaluated under all possible environmental conditions. The phenotype may vary with changes in the environment such as light, temperature, water and nutrient availability, etc. without a change in the genotype of the plant.

### BRIEF DESCRIPTION OF THE SEQUENCES

The sequences shown below in Table 1 conform with 37 C.F.R. §1.821-1.825 (“Requirements for Patent Applications Containing Nucleotide Sequences and/or Amino Acid Sequence Disclosures—the Sequence Rules”).

TABLE 1
List of nucleotide sequences of primers, along
with description as used for Amplified Fragment
Length Polymorphism (AFLP) analysis,
distinguishing ″Thaya″ from other varieties
of catmint.

| SEQ | | |
|---|---|---|
| ID | NUCLEOTIDE SEQUENCE | DESCRIPTION |
| 1 | 5′ GAC GAT GAG TCC TGA G 3′ | Adaptor Primer |
| | | (Msel) |
| 2 | 5′ TAC TCA GGA CTC AT 3′ | Adaptor Primer |
| | | (Msel) |
| 3 | 5′ CTC GTA GAC TGC GTA CC 3′ | Adaptor Primer |
| | | (HindIII) |
| 4 | 5′ AGC TGG TAC GCA GTC | Adaptor Primer |
| | TAC 3′ | (HindIII) |
| 5 | 5′ GAC TGC GTA CCA GCT TA 3′ | Pre-amplification |
| | | primer (HindIII) |
| 6 | 5′ GAT GAG TCC TGA GTA AA 3′ | Pre-amplification |
| | | primer (Msel) |
| 7 | 5′ CTG CGT ACC AGC TTA GC 3′ | Amplification |
| | | Primer (HindIII) |
| 8 | 5′ TGA GTC CTG AGT AAA GC 3′ | Amplification |
| | | Primer (Msel) |

### BRIEF DESCRIPTION OF THE DRAWINGS

FIGS. 1a and 1b contains AFLP chromatograms showing the region of amplified DNA fragments with sizes between 240 and 290 by (base pairs) using SEQ IDs 7 and 8. The chromatograms were derived from processing of PCR reactions using an Applied Biosystems (Foster City, Calif.) Prism 3130XL Genetic Analyzer. The chromatograms were displayed and analyzed using Peak Scanner software (Applied Biosystems, Foster City, Calif.). Varieties in FIG. 1a are labeled “Thaya” and A, B and C (unrelated varieties).

FIG. 2 shows views of the high-oil yielding variety Nepeta cataria var. “Thaya”, wherein Panel A (A) is Leaf, Panel B (B) is flowering spike, Panels C and D (C), (D) are close-ups of flowers.

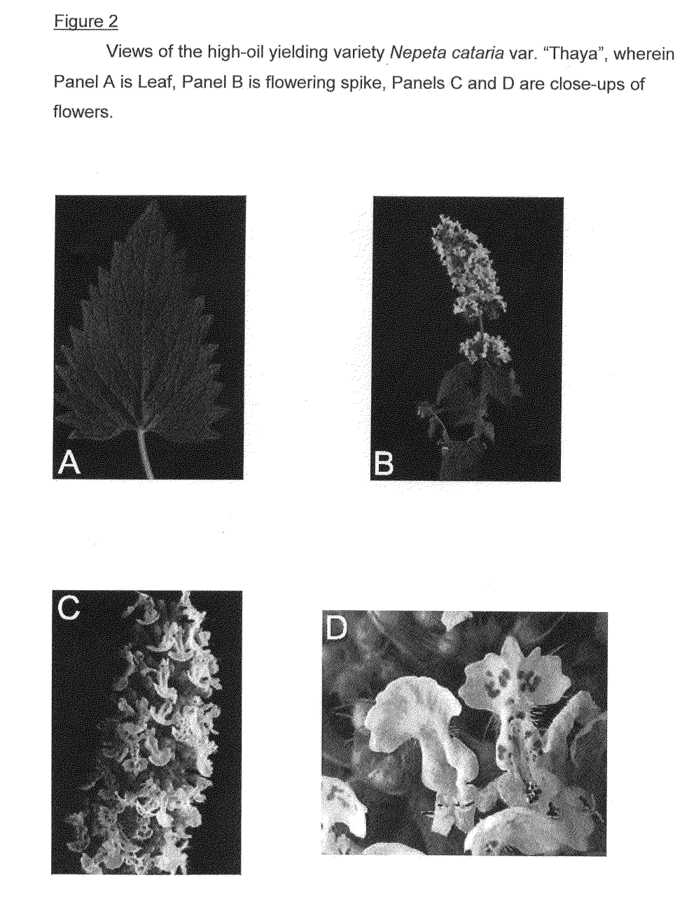

### DETAILED DESCRIPTION

The catmint plant disclosed herein was developed in an effort to cultivate a catmint variety superior in oil content to existing varieties. Thus, a novel and multi-utility, vigorously growing, robust catmint plant Nepeta cataria L. var. “Thaya” was selected from glasshouse-grown populations of accessions of catmint seeds from commercial sources.

Plants were grown from 13 seed accessions (20 plants per group). Specific nepetalactone content in leaf material of these plants was measured as an indicator of essential oil content. During sampling, 42 plants were identified as having nepetalactone yield higher than the average. At flowering, aerial biomass of individual plants was harvested, and the oil was solvent-extracted and analyzed. Percent nepetalactone yield was used as an indicator of essential oil yield, and the 13 highest yielding plants were selected for further study.

Each of these 13 plants was increased in number using cuttings (asexual propagation) and transferred to field plots. Plants from replicate field plots of each line_were harvested at maturity, and oil was extracted by steam distillation. The field plots for the cultivar henceforth termed “Thaya” yielded more oil than that of unimproved commercially available seed in this trial (0.15 vs 0.11%). Some of the retained plants were allowed to self-fertilize in the glasshouse, and the seed collected was used to sow field test plots in a subsequent growing season. The plants were harvested and processed again as described above. Oil yield on steam distillation was 0.33%.

The unique plant variety obtained as described above has been termed “Thaya”. The variety “Thaya” grows as well as other accessions of the species and can be maintained throughout a growing season, and its shoot mass can be harvested for distillation or for leaf. For extraction of oil, the freshly harvested leaves can be dried in the field and subjected to conventional steam distillation or other methods of extraction. The composition of the essential oil of “Thaya”, as determined by gas chromatograph analysis, is shown in Table 2. For the analysis, essential oil was isolated from harvested plant material by steam distillation, and subjected to gas chromatography. The oil is predominantly composed of nepetalactones, with the most abundant stereoisomer being cis,trans-nepetalactone. A variety description of catmint variety “Thaya” is provided in Table 3.

TABLE 2
Composition of essential oil steam distilled from N. cataria var. “Thaya”.
Data (constituent, wt %) derives from separate analyses of distillations of
three field plots planted with clonal plants. Standard error of the mean
(N = 3) is included in parentheses.

| Oil component | Wt % (SEM) | |
|---|---|---|
| cis,trans-nepetalactone | 63.407 | (6.436) |
| trans,cis-nepetalactone | 6.85 | (3.078) |
| Nepetalic Acid | 6.213 | (2.568) |
| β-caryophyllene | 2.057 | (0.082) |
| dihydronepetalactone | 0.323 | (0.133) |
| cis,cis-nepetalactone | 0.31 | (0.08) |

| | |
|---|---|
| puleganic acid | <1.00 |
| germacrene-D | <1.00 |
| copaene | <1.00 |
| iso-caryophyllene | <1.00 |
| iso-caryophyllene oxide | <1.00 |
| nepetalic acid 1 | <1.00 |
| D-+-camphor | <1.00 |
| R-+-limonene | <1.00 |
| α-caryophyllene oxide | <1.00 |
| cymene | <1.00 |
| cis-β-Ocimene | <1.00 |
| trans-β-Ocimene | <1.00 |
| carvone | <1.00 |
| α-caryophyllene | <1.00 |
| piperitone | <1.00 |

TABLE 3
Variety Description Information for “Thaya”
The following description is based on a mature flowering plant of Nepeta
‘Thaya’ grown in glasshouse conditions in MetroMix 360 soil
substitute with supplemental water and fertilizer as needed.

| | |
|---|---|
| Genus | Nepeta |
| Species | cataria L. |
| Family | Labiatae (Lamiaceae) |
| Common name | Catmint, catnip |
| Growth habit | Erect sturdy main stem, profuse branching |
| Plant Height | 45-90 cm |
| Leaf appearance | Light grey-green, triangular-oval, coarsely |
| | toothed, bearing simple and glandular |
| | trichomes |
| Stem | Square in cross-section, with hairy surface |
| | texture |
| Inflorescence | Small labiate flowers, white spotted with |
| | purple, in clusters forming a compact |
| | flowering spike |
| Fruit/seed | Small (ca. 1500/g), dark brown to black. |
| Fragrance | Aromatic |
| Oil yield | 0.15 to 0.33% of dry weight |

The variety “Thaya” is genetically distinct from other accessions of catmint as determined by its genomic DNA fingerprint profile generated by the Amplified Fragment Length Polymorphism (AFLP) technique. The AFLP profile was generated by use of genomic DNA isolated from the leaf samples of “Thaya” plants and three other catmint lines, essentially as described in “Plant Genotyping: the DNA Fingerprinting of Plants” (R. J. Henry, ed., CAB International, 2001) based on the standard procedure of Vos et al in “AFLP: a New Technique for DNA Fingerprinting” (Nucleic Acids Research, 23 (21):4407-14, 1995). The sequences used as primers in AFLP to distinguish “Thaya” from other lines are described as SEQ IDs 1-8 in Table 1. These include Msel and HindIII adaptor pairs (SEQ IDs 1 and 2; SEQ IDs 3 and 4 respectively), pre-amplification (SEQ IDs 5 and 6), and amplification (SEQ IDs 7 and 8) primers.

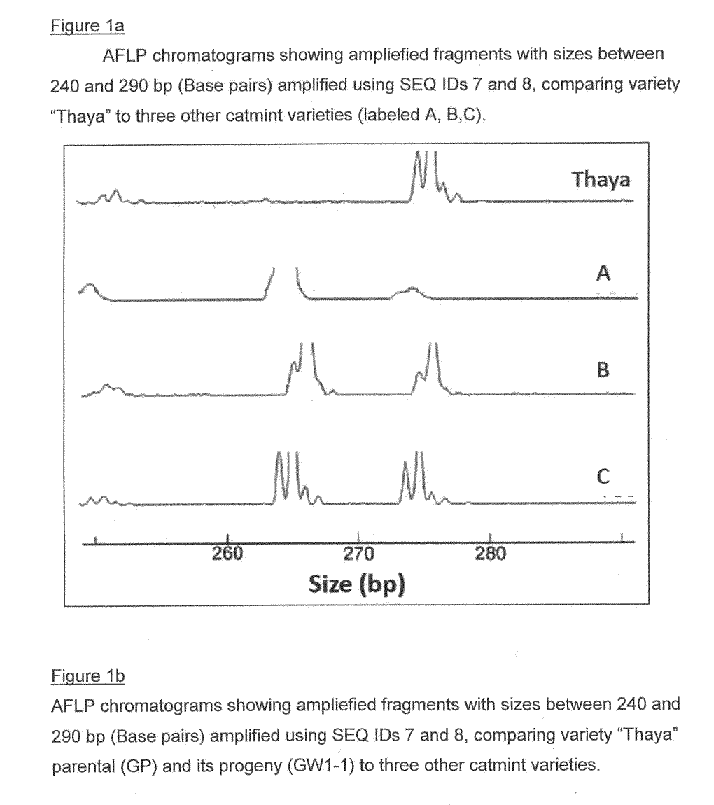

By application of the primer pair described in SEQ IDs 7 and 8, the genetic profile of variety “Thaya” can be distinguished from three other catmint lines. In addition, the parental and one of its asexually reproduced progeny of variety “Thaya” have the same prominent DNA fingerprinting profile with fragments in the 264 bp region missing (Table 4, FIG. 1b). This demonstrates that the DNA fingerprint profile of variety “Thaya” is unique and transferred to asexually reproduced progeny.

TABLE 4
AFLP results of FIG. 1a and 1b, distinguishing “Thaya” from other
catmint varieties. The presence or absence (Yes/No) and number (in
parentheses) of fragments in the region of the chromatogram at
ca. 264 bp are shown for each variety tested.

| Variety “Thaya”* | Variety A | Variety B | Variety C |
|---|---|---|---|
| No (0) | Yes (1) | Yes (3-4) | Yes (4) |

*GP and GW1-1 labeled in FIG. 1b

While the plant disclosed herein has been described as it relates to a specific embodiment, the scope of the present invention is not intended to be limited thereto, and is intended to cover other variations, uses and adaptations that may arise under different environmental conditions.

What is claimed is:

1. A new and distinct form of Nepeta cataria L. plant substantially as shown and described.
